(19) United States
(12) Patent Application Publication
(10) Pub. No.: US 2015/0344951 A1
(43) Pub. Date: Dec. 3, 2015

(54) PRE-IMPLANTATION GENDER SCREENING KIT AND METHOD

(71) Applicant: FERTILITY NEXUS LLC, Laguna Hills, CA (US)

(72) Inventor: Daniel A. Potter, Laguna Beach, CA (US)

(21) Appl. No.: 14/724,023

(22) Filed: May 28, 2015

Related U.S. Application Data
(60) Provisional application No. 62/004,078, filed on May 28, 2014.

(51) Int. Cl. C12Q 1/68

(52) U.S. Cl. CPC C12Q 1/6879

(57) ABSTRACT

A kit and method for determining the gender of a human's or other mammal's pre-implantation embryo with increased accuracy. The method comprises exposing genetic material from one or more cells removed from the embryo to multiple labeled hybridization agents that will detect markers associated with (1) the Y chromosome, but not the X chromosome, (2) the X chromosome, but not the Y chromosome, and (3) both X and Y chromosomes. The gender is determined by detecting the presence or absence of labeled hybridized agents in the sample after washing, or indicates that the test results are not reliable. The kit contains labeled hybridization agents for conducting the pre-implantation gender screening method.

### CROSS-REFERENCE TO RELATED APPLICATIONS

This non-provisional patent application is related to and claims priority from provisional patent application 62/004,078, filed May 28, 2014.

### FIELD OF THE INVENTION

This invention relates generally to determining the gender of an embryo in the period between in vitro conception and implantation. More specifically, the disclosed and claimed subject matter relates to kits and methods of determining the gender of a nascent mammal with a high degree of precision by removing one or more embryonic cells and testing them for the presence of the Y chromosome prior to implantation. The invention also relates to a method of gender-based selection of an embryo prior to implantation.

### BACKGROUND OF THE INVENTION

The first in vitro conception of a human and successful implantation was accomplished in 1978, resulting in the birth of Louise Brown. In vitro fertilization and implantation has since become a conventional practice of medicine that has helped many patients to produce natural offspring. It involves the removal of an oocyte from a female donor and fertilization with sperm from a male donor, to produce a zygote. The zygote continues to grow for several days into a blastocyst, typically 5 or 6 days, before it is implanted in a female, who may be the oocyte donor. In order to increase the likelihood of successful implantation, multiple oocytes are removed from the donor's ovaries, fertilized and implanted.

The creation of a zygote and blastocyst before implantation provides an opportunity to obtain genetic information about the embryo before implantation. (The term embryo is used broadly herein as a reference to the fertilized egg in the zygote, blastocyst and later stages of development. It is not intended to be interpreted narrowly to only the post-implantation period.) A single cell from the embryo provides a complete set of chromosomes for the resulting child. Hence, in vitro fertilization techniques allow access to the entire genome of a nascent baby prior to implantation of the embryo.

The technique of genetic testing of embryonic cells prior to implantation is referred to as Pre-implantation Genetic Diagnosis or PGD or PIGD. Though widely used and accepted in the practice of medicine, the term “diagnosis” is somewhat of a misnomer because it suggests the identification of disease. As an embryo does not exhibit symptoms of disease, a more precise term would be Pre-implantation Genetic Screening or PGS. Alternatively, they can be referred to as Pre-implantation Genetic Profiling or PGP.

With the advent and rapid growth of genetic testing, a multitude of characteristics can be determined from a DNA sample. In the context of in vitro fertilization, this provides the promise of advance notice regarding the child produced by the impending implantation. This currently includes many genetic disorders that can result in miscarriages, early death, or life-long physical and mental challenges for the child.

One characteristic of interest to some prospective parents undertaking in vitro fertilization procedures is the gender of the child. Pre-implantation testing has included testing for gender and selection of specific male or female embryos. Gender of a child is governed by the two gender-related chromosomes. If the cell's genome contains two X-chromosomes the embryo will develop into a female baby. If the genome contains one X-chromosome and one Y-chromosome, the embryo will develop into a male baby.

Methods of gender testing a single cell or small group of cells are known. However, certain challenges arise when applying these methods for gender determination in the short, pre-implantation period. An investigation of the presence of the sex-related chromosomes cannot be conducted on the oocyte before fertilization. The oocyte is the germ cell of a female and therefore can contribute only an X-chromosome to its offspring. Hence, oocytes cannot be selected to determine the gender of the resulting baby.

Sperm, on the other hand, contributes either the X or Y chromosome that determines the gender of the embryo. If an X is contributed, the embryo will have two X chromosomes, and be female. If a Y chromosome is contributed, the embryo will have one X and one Y chromosome, and be male.

It is possible to select the gender of an embryo prior to fertilization by segregating individual sperm cells into cohorts of all X or all Y chromosome-containing sperm cells. However, performing this procedure delays the mixing of the oocytes with the sperm, reduces the virility of the sperm, and decreases the likelihood and/or the number of zygotes successfully obtained by the fertilization step. Thus, selecting gender by separating X chromosome-containing sperm from Y chromosome-containing sperm has substantial disadvantages.

In addition, oocytes are less viable over time than zygotes. It is desirable to fertilize the oocytes with sperm and create a zygote as soon as the oocytes are removed from the donor. Zygotes can be preserved for an indefinite period of time by freezing. This allows for an egg retrieval procedure to provide sufficient embryos for an initial implantation as well as a supply of additional embryos for multiple, later implantations, if necessary.

Gender testing and selection is conducted in the time between creation of the zygotes and implantation in the prospective mother. Testing has to occur after the egg is fertilized and the zygote is created because this establishes the chromosome set for the embryo and ultimately the baby. However, testing also has to occur before implantation as only the selected embryos will be implanted. As mentioned earlier, this window is only 5 to 6 days long.

The relatively short period of time after creation of the zygote and implantation presents challenges for gender testing. During this time, for each potentially implanted embryo, (1) one or more cells need to be removed, (2) the cells need to be tested to determine gender (and other traits conducted in conjunction with gender testing), and (3) the selected embryos are identified, and segregated from the unselected embryos. It is desirable to implant multiple embryos for various reasons including the statistical likelihood of success and, if successful, the desire for multiple births (twins, triplets). Fewer than 50% of the tested embryos can be expected to be selected (because gender disqualify about 50% and other factors, such as detected chromosomal abnormalities, may reduce the number even further). Therefore, this procedure typically is conducted on a pre-determined number of multiple embryos.

The embryo grows from a single cell to a blastocyst during the 5-6 pre-implantation time period. The sampling process needs at least one cell from the developing embryo for testing to be conducted. The removal of a cell can be conducted without injuring the embryo of significantly slowing its growth. However, taking too many cells too early is not without risks as the removed tissue comprises a larger proportion of the total embryo. Taking one cell presents less of a risk that taking multiple cells, particularly at a relatively early point in the 5 to 6 day period. Moreover, conducting the biopsy later provides less risk if conducted later in the period, particularly if multiple cells are removed.

There is a trade-off between number of cells that can be tested per embryo versus the amount of time remaining to conduct the tests, obtain the results and select the gender-desirable embryos. Sampling at an early point in the period provides more time to obtain the results, but reduces the number of cells that can be safely sampled. Early in the 5 to 6 day period only a single cell can be sampled without prejudicing the viability of the embryo.

Even on the third day after conception, typically no more than one or two cells can be safely biopsied. While this provides three days before implantation, it still only provides the option to biopsy one or two cells for testing. The accuracy of the test is lowered by having fewer cells available per embryo—the three day window does not allow for additional testing on the removed cells to improve accuracy.

Conversely, more cells, about 3 to 5, can be sampled from each embryo by conducting a biopsy on day 5. The additional number of cells provides more robust and, therefore, more accurate test signals and results. Sampling at a later point in the period provides the potential to sample multiple cells without significantly impacting viability of the embryo. Using multiple cells increases the likelihood of an accurate gender identification. However, it leaves less time for steps of testing, obtaining results and selecting embryos based on the results. Thus, it is desirable to sample multiple cells per embryo to increase accuracy, but only if the test procedure can be accomplished in the shorter period of time left before implantation.

The conventional method currently used for PGS is Comparative Genomic Hybridization or CGH. CGH is a molecular cytogenetic method for analyzing ploidy copy number variations in DNA samples by comparing an unknown sample with a known reference sample. The technique compares DNA samples from two sources to detect differences between chromosomal compliments.

CGH can be used to determine gender as well as other chromosome or ploidy-level abnormalities. For example, CGH can detect the presence of two X chromosomes, indicating a female, or the absence of two X and therefore the presence of one X chromosome and one Y chromosome, indicating a male. It is also often used to detect aneuploidy, i.e., numerical chromosomal disorders, such as Turner Syndrome, where a person is born with only a single, X chromosome.

Other aneuploidy disorders found by conducting CGH include the presence of three instead of two chromosomes. Down Syndrome, also known as Trisomy 21, is the presence of three copies of chromosome 21 instead or two. In addition, CGH is used to detect the presence of three sex-chromosomes. For example, Klinefelter Syndrome is indicated where a person has two Xs and one Y; 47,XYY Syndrome, or Super Male syndrome is indicated by the presence of one X and two Y chromosomes; and Triple X or 47,XXX Syndrome is the presence of three X and no Y chromosomes.

More recently, the CGH technique has been practiced with DNA microarrays. This newer test procedure is referred to an array CGH or aCGH. DNA from a reference or control sample is compared with DNA from an unknown sample such as samples from a patient seeking a diagnosis. Initially, large insert genomic DNA clones, such as Bacterial Artificial Chromosome or BAC, were used to produce arrays. Later, Polymerase Chain Reaction or PCR techniques were employed for whole genome amplification.

Array CGH techniques allow for gender testing as they simultaneously determine gender and ploidy abnormalities, which is particularly welcome given the time constraints for completion of PGS. The test provides reasonably accurate results for gender determination—typically 95% or greater. However, for in vitro fertility patients who have invested substantial time, physical effort and financial resources with the hope of natural childbirth, greater accuracy is desired. For those expectant parents who have a strong preference for a specific gender, a single test failure can be very disappointing. Increased accuracy will provide fewer disappointments every year.

### SUMMARY OF THE INVENTION

The present invention provides a kit and method for improving the accuracy of pre-implantation gender determination as part of PGS. The method is practiced in conjunction with and simultaneous to conventional aCGH testing methods. It employs steps that amplify and detect specific DNA sequences or markers that are unique to one or both of the X chromosome and Y chromosome. The presence or absence of the Y chromosome is confirmed with greater specificity and fewer errors.

The inventive kit and method includes a check to determine if the absence of a signal associated with the presence of the Y chromosome correctly reflects the absence of the chromosome in the sample genome, or a failure of the test to detect any signal whatsoever. In the event of an absence in the signal, the failure to amplify in the presence of the Y chromosome because of a test error is ruled out by a marker that corresponds to the presence of a sequence known to be present on both the X and Y chromosomes. The detection of the both markers indicates the presence of the Y chromosome and a male embryo, the presence of a single marker indicates the absence of a Y chromosome and a female embryo, and the absence of both indicates the failure of the test to make a reliable determination.

### DETAILED DESCRIPTION

The kit and testing method contemplated herein utilizes a combination of gender determining techniques to obtain increased accuracy. The increase in accuracy is achievable with the sampling of one or two cells at a relatively early point of the 5 to 6 day period before implantation. It is also achievable with the use of a larger number of sampled cells, but late in the period, when the turn-around time for results is relatively short.

The inventive kit and method utilizes the conventional PGS technique utilizing array CGH. This method is a whole-genome comparison with a reference sample. The accuracy of this technique is increased when combined with PCR amplification and detection techniques for specific genetic sequence markers known to be present either (1) on the Y chromosome, but not the X chromosome, (2) on the X chromosome, but not the Y chromosome, and (3) on both X and Y chromosomes. Testing for the presence of markers falling within all three of these categories provides the desired information to determine whether the sample contains only X chromosomes, indicating that the cell came from an embryo for a female baby, or contains an X chromosome and a Y chromosome, indicating that the cell came from an embryo for a male baby.

Use of the third category of markers will avoid a false identification of a male embryo as a female. If only marker categories (1) and (2) were used, the failure of the Y marker to be detected for some technical reason other than the absence of the Y chromosome would provide a false result that the embryo is a female. Use of an additional marker category (3) will necessarily detect the presence of the X chromosome and provide a signal. If that signal fails, then it will indicated that the test failed to provide a reliable result of the absence of the Y chromosome, and the status of the embryo as a female. If category (1) marker is not detected, and categories (2) and (3) are detected, this will indicate a reliable result that the test accurately determined the absence of the Y chromosome for the tested embryo. If all three categories of markers are detected in the sample, the test reliably indicates that the tested embryo is male.

The process for array genomic hybridization for determining gender is well known to those working in the PGS art. The amplification and detection of chromosome specific markers as described herein is not generally used by or familiar to those working in the art. Such techniques, however, have been utilized by phylogeographic researchers to investigate population history. For example, Underhill, P. A., et al., The Phylogeography of Y Chromosome Binary Haplotypes and the Origins of Modern Human Populations, Ann. Hum. Genet. (2001), 65, 43-62, presents a phylogeographic reconstruction based on the presence of certain polymorphisms in current, geographically distinct human populations. The researchers used markers on a non-recombining portion of the Y-chromosome as evidence of migrations, colonizations and differentiations over time.

Similarly, Cengiz Cinnioglu et al., Excavating Y-chromosome Haplotype Strata in Anatolia, Hum. Genet. (2004) 114: 127-148, describes the use of DHPLC methodology to detect certain polymorphic markers in human populations. Again this information was used for the characterization of human populations and to gain insights into the historical relationships of currently distinct groups, and not for PGS. However, the techniques described to detect markers on the Y chromosome can be used for the purposes described above for the present invention. The teachings of those two scientific articles are incorporated by reference herein.

The process contemplated herein is to remove one or more cells from a newly-created embryo. The number of cells is limited by the age of the embryo. If the embryo is only two or three days old, a relatively small number can be removed without risk to the embryo. If older, more cells may be removed for testing.

The genetic material is subsequently removed from the embryonic cells by known techniques. This makes the genetic material including the chromosomes available in solution to be exposed to reagents. The genetic material from as little as a single cell can be amplified to greater, easier to measure amounts by the use of the polymerase chain reaction, which is a known technique. This provides a greater volume of genetic material for hybridization in the subsequent testing and increases the signals for determining the presence of absence from the labels in the sample.

Once the genetic material from the cell or cells is released into solution and amplified, the sample is ready for testing. Testing is accomplished by exposing the sample to labeled hybridization agents. The exposure of the labeled hybridization agents can be accomplished together, in a simultaneous manner, or seriatum, i.e., one hybridization step at a time. If conducted in sequence, no particular order is necessary to accomplish the result of the method.

Each agent has a distinct label so that its presence in the sample, after hybridization and filtering/washing can be determined. Such filtering/washing steps are known in the art. The presence of a label in the sample will, therefore, indicate that hybridization has occurred between the particular agent associated with the label and the particular marker to which the agent binds.

The preferred embodiment of the inventive kit and method utilizes specific sequences for the three category of sequences. Category (1), the Y chromosome markers that indicate the presence of the Y chromosome, but not the X chromosome includes three specific markers on the Y chromosome: M219, M221 and M224. Category (2), the X chromosome markers that indicate the presence of the X chromosome, but not the Y chromosome includes one specific marker on the Y chromosome: X-STS-1. Category (3), the chromosome markers that are found on both X and Y chromosomes and therefore indicate the presence of either the X chromosome or the Y chromosome include one specific marker: X353.

The reagents for conducting the inventive method can be packaged together as a kit for the purpose of being used to practice the inventive method. The package would include first, second and third labeled hybridization agents that would hybridize to the X and Y chromosomes. For example, one of the labeled hybridization agents in the kit would selectively hybridize to one or more of the markers M219, M221 and M224. These markers are associated with the Y chromosome, but not the X chromosome.

Similarly, the kit could include a second labeled hybridization agent that selectively hybridizes to marker S-STS-1. This marker is associated with the X chromosome, but not the Y chromosome. The kit could also include a third labeled hybridization agent that selectively hybridizes to marker X353. This marker is found on both X and Y chromosomes and associated, therefore, with the presence of either or both chromosomes. If no hybridization occurs with the agent associated with this marker, then the test is faulty and the results unreliable.

The kit may also include materials, reagents, containers, reactors and other components for removing a cell from an embryo or for making the genetic material from an embryonic cell available for testing in solution. For example, certain reagents that are used to release of genetic material from the cell could be provided as part of the kit. Similarly, reagents or other materials or components used for polymerase chain reaction to amplify the volume of genetic material could be included in the kit.

The kit could further include a device for determining which of the labeled hybridization agents hybridized to the embryonic cellular sample to which it was exposed. Such a device could include a label reader appropriate for detecting the particular labels. Labels may be color coded, for example, and the label detector/reader would be an optical device to detect the presence of specific wavelengths of light associated with each selective hybridization agent. A detected telltale wavelength would indicate the presence of the label, and associated marker, in the sample.

Alternatively, the kit could include additional reagents that react with the labels, thereby indicating the presence or absence of the markers in the sample. For example, reagents could be provided that react with labels on the first labeled hybridization agent, but not the labels on the second and third labeled hybridization agents. A different reagent that selectively reacts with the second labeled agent could be provided in the kit. A third reagent that selectively reacts with the third labeled agent could also be provided. In this way, the presence or absence of reactions with those agents could be used to determine which markers are present in the embryonic chromosomal sample of interest. Hence, reactivity of these agents to the sample would be associated with the absence or presence of the Y chromosome, and the gender of the embryo, with a high degree of accuracy, or that the test was faulty and does not provide reliable results.

The kit could also include one or more containers, tubes or reactors for preparing the sample and/or exposing the sample to the reagents. In addition, instructions for the use of the various components and reagents could be provided in the kit for the practice of the pre-implantation gender screening method contemplated herein. The kit would be a self-contained unit to practice some or all of the steps discussed above.

The method could be practiced by skilled professionals in controlled medical testing laboratories or under certain circumstances and arrangements by lesser skilled persons in other areas, locations or places. For example, there may be an application of the method appropriate for use by animal researchers or zoologists, or for animal husbandry, in which human medical standards would not apply. A kit could be created that is specific for such an application.

It is understood that other markers and reagents can be used to practice the kit and method of the invention described herein. Other markers in each of the categories can be used, if used together, will provide the necessary information to increase the accuracy over conventional PGS techniques. Alternatively, the kit and method can be practiced with some specific markers described above in addition with other markers falling into the same of other categories, i.e., selective for the Y or X chromosome, or both.

The kits and methods described above are examples of kits and methods falling within the scope of the subject matter described herein and are not intended to limit the scope of the invention as recited in the following claims. Specific details, even if helpful to the understanding and practice of the subject matter, are not intended to be incorporated into the claims unless specifically recited in the claims.

What is claimed is:

1. A method of determining the gender of a pre-implantation mammalian embryo by sampling one or more embryonic cells, the method comprising the steps of:
removing at least one cell from an embryo, the cell containing genetic material;
exposing the genetic material to a first labeled hybridization agent that will hybridize to a Y chromosome, but not an X chromosome;
exposing the genetic material to a second labeled hybridization agent that will hybridize to the X chromosome, but not the Y chromosome;
exposing the genetic material to a third labeled hybridization agent that will hybridize to both the X chromosome and the Y chromosome; and,
determining which labeled hybridization agents hybridized on the X and Y chromosomes.

2. The gender determination method of claim 1 in which the genetic material is simultaneously exposed to all three of the labeled hybridization agents.

3. The gender determination method of claim 1 in which the genetic material is exposed to each labeled hybridization agent seriatim.

4. The gender determination method of claim 1, further comprising the step of conducting array comparative genomic hybridization on the cell or cells to determine the absence or presence of the Y chromosome.

5. The gender determination method of claim 4, wherein the embryo is confirmed as male if (1) the comparative genomic hybridization indicates the presence of the Y chromosome, and (2) hybridization of the first, second and third labeled agents is detected.

6. The gender determination method of claim 4, wherein the embryo is confirmed as a female if (1) the comparative genomic hybridization indicates the absence of the Y chromosome, and (2) hybridization of the second and third labeled hybridization agents is detected.

7. The gender determination method of claim 4, wherein the detection of the hybridization of only the second labeled hybridization agent indicates that the test results are not reliable.

8. The gender determination method of claim 1, wherein the mammalian embryo is human.

9. The gender determination method of claim 1, wherein the first hybridization agent hybridizes to the marker selected from the group consisting of M219, M221, M224 and combinations thereof.

10. The gender determination method of claim 1, wherein the second hybridization agent hybridizes to the marker S-STS-1.

11. The gender determination method of claim 1 the third hybridization agent hybridizes to the marker X353.

12. A method of determining the gender of a pre-implantation human embryo by sampling one or more cells, the method comprising the steps of:
removing at least one cell from an embryo, the cell containing genetic material;
conducting array comparative genomic hybridization on the cell or cells to determine the absence or presence of the Y chromosome;
exposing the genetic material to a first hybridization agent that will hybridize to a marker selected from the group consisting of M219, M221, M224 or combinations thereof;
exposing the genetic material to a second labeled hybridization agent that will hybridize to marker S-STS-1;
exposing the genetic material to a third labeled hybridization agent that will hybridize to marker X353; and,
determining which hybridization agents hybridized on the genetic material.

13. The gender determination method of claim 12 in which the genetic material is simultaneously exposed to all three of the labeled hybridization agents.

14. The gender determination method of claim 12 in which the genetic material is exposed to each labeled hybridization agent seriatim.

15. The gender determination method of claim 12, wherein the embryo is confirmed as male if (1) the comparative genomic hybridization indicates the presence of the Y chromosome, and (2) hybridization of the first, second and third labeled agents is detected.

16. The gender determination method of claim 12, wherein the embryo is confirmed as a female if (1) the comparative genomic hybridization indicates the absence of the Y chromosome, and (2) hybridization of the second and third labeled hybridization agents.

17. The gender determination method of claim 12, wherein the detection of the hybridization of only the second labeled hybridization agent indicates that the test results are not reliable.

18. A kit for determining the gender of a mammalian embryo prior to implantation of the embryo by testing a cellular sample that has been removed from the embryo, the kit comprising:
a first labeled hybridization agent capable of selectively hybridizing to a Y chromosome and not an X chromosome of the cellular sample;
a second labeled hybridization agent capable of selectively hybridizing to the X chromosome and not the Y chromosome of the cellular sample; and,
a third labeled hybridization agent capable of hybridizing to both the Y chromosome and the X chromosome of the cellular sample.

19. The kit for determining the gender of a cellular sample of claim 18 wherein the first labeled hybridization agent is capable of hybridizing to a marker selected from the group consisting of M219, M221, M224 and combinations thereof.

20. The kit for determining the gender of a cellular sample of claim 18 wherein the second labeled hybridization agent is capable of hybridizing to marker S-STS-1

21. The kit for determining the gender of a cellular sample of claim 18 wherein the third labeled hybridization agent is capable of hybridizing to marker X353.
